(19) United States
(12) Patent Application Publication
(10) Pub. No.: US 2014/0100285 A1
(43) Pub. Date: Apr. 10, 2014

(54) COMPOSITIONS OF PERCUTANEOUS ADMINISTRATION OF PHYSIOLOGICALLY ACTIVE AGENTS

(71) Applicants: Stephan Meyer, Clarens (CH); Aurelie Lang, Gex (FR)

(72) Inventors: Stephan Meyer, Clarens (CH); Aurelie Lang, Gex (FR)

(73) Assignee: Novartis AG, Basel (CH)

(21) Appl. No.: 14/119,634

(22) PCT Filed: May 24, 2012
(86) PCT No.: PCT/EP2012/059670

§ 371 Date: Nov. 22, 2013

(30) Foreign Application Priority Data
May 26, 2011 (EP) 11167614.4

(51) Int. Cl. A61K 9/70; A61K 47/32

(52) U.S. Cl. CPC A61K 9/7015; A61K 47/32
USPC 514/567; 514/772.6

(57) ABSTRACT

Compositions intended for the percutaneous administration of physiologically active agents, e.g. drugs or a veterinary agents, are disclosed. Said compositions are characterized forming a non-sticky, very flexible film that ‘has excellent long term efficacy.

The present invention relates to compositions intended for the percutaneous administration of physiologically active agents, in particular pharmaceutically active compounds (but also e.g. agents like nicotine or veterinary drugs). Throughout this document, “percutaneous” is intended to mean any route of administering a physiologically active agent onto, into or through the skin of a subject so as to achieve one or more of a topical, local or systemic physiological effect. Local treatment of the skin is intended to also include, for example, applying such compositions to the ear, e.g. for treating otitis with e.g. antibiotics. More specifically, the invention relates to percutaneous pharmaceutical and veterinary, especially pharmaceutical, compositions with improved long term efficacy.

More specifically, the invention concerns compositions which when applied to the skin rapidly form a durable, flexible film comprising one or more physiologically active agents, in particular pharmaceutically active substances.

Such film forming compositions are known in the art. However, said known film forming compositions (see e.g. U.S. Pat. No. 6,211,250) suffer from various disadvantages, e.g. when applied to the skin, they may be sticky for a while, meaning that they may adhere e.g. to clothes for some time.

The present invention overcomes said problems by providing a film forming composition, preferably in gel form, that rapidly forms a non-sticky, durable, very flexible film.

Thus, the present invention relates to a composition for percutaneous administration of a physiologically active agent comprising

(a) ethyl acrylate/methyl methacrylate copolymer,
(b) ethanol, isopropanol or a mixture thereof,
(c) water, and
(d) at least one physiologically active agent.

Ethyl acrylate/methyl methacrylate (EA/MMA) copolymers typically act as film forming polymer in the compositions. EA/MMA copolymers (a) when combined with ethanol and/or isopropanol (b) and water (c), have been identified as beneficial film forming polymers typically providing a gel. The resulting compositions of the invention can, for example, be packaged in tubes like any conventional topical semi-solid form, or applied to the skin via special devices developed for being able to spray gel-like compositions, e.g. Tru-Spray®. The viscosity of the formulation can be adjusted to the optimal viscosity desired for a particular package, for example, by adapting the weight ratio of (a):(b), i.e. of ethyl acrylate/methyl methacrylate copolymer: ethanol/isopropanol. By increasing the amount of ethanol/isopropanol, the viscosity of the formulation is decreased and vice versa. When using very low amounts of (a) [e.g. 2-5%, preferably 4-5%] and very high amounts of (b) [e.g. 70-90%], viscous liquids—instead of gels—may be obtained (see Examples 10-11). Those form beneficial films on the skin as well and thus are also within the scope of the present invention. They may be applied to the skin, for example, as a spray.

All percentages given hereinbefore and hereinafter are weight-%, unless indicated otherwise.

Ethyl acrylate/methyl methacrylate (EA/MMA) copolymers (a) can be used in pure form but also e.g. in the form of a dispersion or a solution. Preferably, they are used in the form of an aqueous suspension, for example as a product of the Eudragit® NE series, e.g. Eudragit® NE 30 D or Eudragit® NE 40 D, or Eudragit NM 30 D, all provided e.g. by the company Evonik Industries. For example, Eudragit® NE 40 D includes 40% of EA/MMA copolymer and 60% of water. Typically, the compositions of the invention contain EA/MMA copolymers (a)—calculated in pure form, i.e. by excluding e.g. any solvents that may be present—in an amount of at least 2%, preferably at least 5%, more preferably at least 10%, or in an amount of from 2 up to 30%, in particular of from 5 up to 25%.

The solvent or mix of solvents (b) used, i.e. ethanol and/or isopropanol, are volatile so that they quickly evaporate after administration and allow formation of a film on the skin. Preferred as (b) are isopropanol or a mixture of isopropanol and ethanol, and in particular isopropanol. Typically, the compositions of the invention contain the solvent or mix of solvents (b) in an amount of at least 30%, preferably at least 40%, or in an amount of from 35% up to 90%, or of from 35% up to 80%, or of from 40 up to 70%.

Typically, the compositions of the invention contain EA/MMA copolymers (a)—calculated in pure form—and component (b) [=ethanol and/or isopropanol] in a weight ratio of from 1:1 up to 1:45, preferably 1:1 up to 1:25, especially 1:1 up to 1:15, and in particular of from 1:1 up to 1:10.

Water (c) typically is either incorporated by using aqueous dispersions of component (a), e.g. Eudragit® NE 40 D, and/or is added separately to the composition. Typically, the compositions of the invention contain water (c) in an amount of at least 1%, preferably at least 5%, more preferably at least 10%, especially at least 15%, or of from 1% up to 50%, preferably of from 5% up to 40%, and in particular of from 15% up to 35%.

Physiologically active agents (d) are, in particular, pharmaceutically active compounds but also include e.g. agents helping in smoking cessation e.g. nicotine, or veterinary drugs.

Used as physiologically active agents (d) can be any pharmaceutically- or veterinarily-active substance suitable for percutaneous delivery. Even physiologically active agents that are normally delivered by the oral, parenteral or rectal route, may come into consideration.

As far as the physiologically active agents (d) are capable of forming physiologically acceptable salts, prodrugs, hydrates or solvates, the latter are included by naming (d) in free, neutral form.

Examples of physiologically active agents (a) are:

Cardioactive medications, for example, organic nitrates such as nitroglycerine, isosorbide dinitrate, and isosorbide mononitrates; quinidine sulfate; procainamide; thiazides such as bendroflumethiazide, chlorothiazide, and hydrochorothyazide; nifedipine; nicardipine; adrenergic blocking agents, such as timolol and propranolol; verapamil; diltiazem; captopril; clonidine and prazosin.

Androgenic steroids, such as testosterone, methyltestosterone and fluoxymesterone.

Estrogens, such as conjugated estrogens, esterified estrogens, estropipate, 17beta estradiol, 17beta-estradiol valerate, equilin, mestranol, estrone, estriol, 17beta-ethinyl estradiol, and diethylstilboestrol. Progestational agents, such as progesterone, 19-norprogesterone, norethindrone, norethindrone, acetate, melengestrol, chlormadinone, ethisterone, medroxyprogesterone acetate, hydroxyprogesterone caproate, ethynodiol diacetate, norethynodrel, 17alpha hydroxyprogesterone, dydrogesterone, dimethisterone, ethinylestrenol, norgestrel, demegestone, promegestone, and megestrol acetate.

Drugs having an action on the central nervous system, for example sedatives, hypnotics, antianxiety agents, analgesics and anaesthetics, such as chloral, buprenorphine, naloxone, haloperidol, fluphenazine, pentobarbital, phenobarbital, secobarbital, codeine, fentanyl, and nicotine.

Local anesthetics, e.g. lidocaine, tetracaine, dyclonine, benzocaine, dibucaine, methocaine, procaine, mepivacaine, bupivacaine, etidocaine or prilocaine.

Nutritional agents, such as vitamins, e.g. Vitamin B12, essential amino acids, and essential fats.

Anti-inflammatory agents, such as steroids, e.g. hydrocortisone, desonide, cortisone, dexamethasone, fluocinolone, triamcinolone, medrysone, prednisolone, flurandrenolide, prednisone, halcinonide, methy prednisolone, flurandrenolide, prednisone, halcinonide, methylprednisolone, fludrocortisone, corticosterone, paramethasone, betamethasone; and non-steroidal anti-inflammatory drugs, e.g. diclofenac, ibuprofen, naproxen, fenoprofen, fenbufen, flurbiprofen, indoprofen, ketoprofen, suprofen, indomethacin, piroxicam, aspirin, salicylic acid, diflunisal, methyl salicylate, phenylbutazone, sulindac, mefenamic acid, meclofenamate sodium or tolmetin.

Anti-inflammatory agents that are often used, inter alia, in veterinary medicine, e.g. triamcinolone, betamethasone, dexamethasone, isoflupredone, hydrocortisone or prednisolone.

Antihistamines, such as dimethindene, diphenhydramine, dimenhydrinate, perphenazine, triprolidine, pyri amine, chlorocyclizine, promethazine, carbinoxamine, tripelennamine, brompheniramine, hydroxyzine, cyclizine, meclizine, clorprenaline, terfenadine, and chlorpheniramine.

Anti-emetic agents, e.g. scopolamine.

Anti-hypertensive drugs, e.g. clonidine.

Respiratory agents, such as theophilline and beta2-adrenergic agonists such as albuterol, terbutaline, metaproterenol, ritodrine, carbuterol, fenoterol, quinterenol, rimiterol, solmefamol, soterenol, and tetroquinol.

Sympathomimetics, such as dopamine, norepinephrine, phenylpropanolamine, phenylephrine, pseudoephedrine, amphetamine, propylhexedrine, and epinephrine. Miotics, e.g. pilocarpine. Cholinergic agonists, such as choline, acetylcholine, methacholine, carbachol, bethanechol, pilocarpine, muscarine, and arecoline.

Antimuscarinic or muscarinic cholinergic blocking agents such as atropine, scopolamine, homatropine, methscopolamine, homatropine methylbromide, methantheline, cyclopentolate, tropicamide, propantheline, anisotropine, dicyclomine, and eucatropine. Mydriatics, such as atropine, cyclopentolate, homatropine, scopolamine, tropicamide, eucatropine and hydroxyamphetamine.

Psychic energizers, e.g. 3-(2-aminopropyl)indole or 3-(2-aminobutyl) indole.

Antibiotics, e.g. clindamycin, erythromycin, tetracycline, penicillin, chloramphenicol, sulfacetamide, sulfamethazine, sulfadiazine, sulfamerazine, sulfamethizole or sulfisoxazole.

Antibiotics that are often used, inter alia, in veterinary medicine, e.g. benzylpenicillin, methycyllin, ampillicin, amoxicillin, streptomycin, neomycin, tetracyclines, chloramphenicol, erythromycin, griseofulvin, thiostrepton, florfenicol, enrofloxacin, bacitracin, gentamycin, polymyxin B, chloramphenicol, marbofloxacin or framecytin.

Antiparasitizides that are often used, especially in veterinary medicine, e.g. malachite green, methylene blue, chloramine T or B, emmamectin benzoate or alpha-cypermethrin.

Anthelmintics that are often used, inter alia in veterinary medicine, e.g. arecoline, ivermectin, praziquantel, mebendazole or thiabendazole.

Antipsoriatic agents, e.g. calcipotriol or calcipotriol/betamethasone combinations.

Antivirals, e.g. penciclovir, acyclovir or idoxuridine.

Anti-acne agents, e.g. benzoyl peroxide.

Dermatological agents, such as vitamins A and E.

Humoral agents, such as the prostaglandins, natural and synthetic, for example PGE1, PGF2alpha, and PGF2alpha, and the PGE1 analog misoprostol.

Antispasmodics, such as atropine, methantheline, papaverine, cinnamedrine, and methscopolamine.

Antidepressant drugs, such as selegiline, isocarboxazid, phenelzine, tranylcypromine, imipramine, amitriptyline, trimipramine, doxepin, desipramine, nortriptyline, protriptyline, amoxapine, maprotiline, and trazodone.

Drugs for the treatment of Alzheimer's disease, e.g. rivastigmine.

Drugs for the treatment of urinary incontinence, e.g. oxybutynin.

Contraceptives, e.g. a mixture of norelgestromin and ethinyl estradiol.

Anti-diabetics, such as insulin, and anticancer drugs such as tamoxifen and methotrexate.

Anorectic drugs, such as dextroamphetamine, methamphetamine, phenylpropanolamine, fenfluramine, diethylpropion, mazindol, and phentermine.

Anti-allergenics, such as antazoline, methapyrilene, chlorpheniramine, pyrilamine and pheniramine.

Tranquilizers, such as reserpine, chlorpromazine, and antianxiety benzodiazepines such as alprazolam, chlordiazepoxide, clorazeptate, halazepam, oxazepam, prazepam, clonazepam, flurazepam, triazolam, lorazepam and diazepam.

Antipsychotics, such as thiopropazate, chlorpromazine, triflupromazine, mesoridazine, piperacetazine, thioridazine, acetophenazine, fluphenazine, perphenazine, trifluoperazine, chlorprathixene, thiothixene, haloperidol, bromperidol, 1oxapine, and molindone.

Decongestants, e.g. xylometazoline, oxymetazoline, phenylephrine, ephedrine or naphazoline.

Antipyretics, e.g. acetylsalicylic acid or salicylamide.

Antimigraine agents, e.g. dihydroergotamine or pizotyline.

Drugs for treating nausea and vomiting, such as chlorpromazine, perphenazine, proclorperazine, promethazine, triethylperazine, triflupromazine, and trimeprazine.

Anti-malarials, such as the 4-aminoquinolines, alpha-aminoquinolines, chloroquine, and pyrimethamine.

Anti-ulcerative agents, such as misoprostol, omeprazole, and enprostil.

Peptides and proteins, such as drugs for Parkinson's disease, spasticity, and acute muscle spasms, such as levodopa, carbidopa, amantadine, apomorphine, bromocriptine, selegiline (deprenyl), trihexyphenidyl hydrochloride, benztropine mesylate, procyclidine hydrochloride, baclofen, diazepam, dantrolene, insulin, erythropoietin and growth hormone.

Anti-estrogen or hormone agents, such as tamoxifen or human chorionic gonadotropin.

Nucleotides and nucleic acids (e.g. DNA).

Antifungals, e.g. terbinafine, naftifine, butenafine, bifonazole, clotrimazole, econazole, isoconazole, ketoconazole, miconazole, oxiconazole, sertaconazole, sulconazole, tioconazole, tolnaftate, terconazole, amorolfine, ciclopirox or undecylenic acid.

Antifungals that are often used, inter alia, in veterinary medicine, e.g. fluconazole, ketoconazole, isoconazole, miconazole, Amphotericin B, flucytosine, terbinafine, nystatin, thiabendazole or clotrimazole.

The physiologically active agents (d) can be present in the composition in different forms, depending on which form yields the optimum delivery characteristics. For example, they can be in its free base or acid form, or in the form of salts, esters, or any other pharmacologically acceptable derivatives, or e.g. as components of molecular complexes.

Preferred physiologically active agents (d) are nicotine, lidocaine, hydrocortisone, diclofenac, ibuprofen, naproxen, indomethacin, piroxicam, methyl salicylate, phenylbutazone, mefenamic acid, betamethasone, dimetindene, scopolamine, benzoyl peroxide, calcipotriol, penciclovir, acyclovir, vitamin A, vitamin E, xylometazoline, oxymetazoline, phenylephrine, ephedrine, naphazoline, terbinafine, naftifine, butenafine, bifonazole, clotrimazole, econazole, isoconazole, ketoconazole, miconazole, oxiconazole, sertaconazole, sulconazole, tioconazole, tolnaftate, terconazole, amorolfine, ciclopirox, undecylenic acid, clonidine, testosterone, nitroglycerin, selegiline, rivastigmine and oxybutynin.

Especially preferred physiologically active agents (d) are nicotine, lidocaine, hydrocortisone, diclofenac, dimetindene, scopolamine, benzoyl peroxide, calcipotriol, penciclovir, acyclovir, xylometazoline, terbinafine, tolnaftate, clotrimazole and rivastigmine.

In a particular embodiment of the invention, the physiologically active agents (d) are selected from the group consisting of pain-relieving pharmaceutically active substances, antihistamines, antifungals and nicotine.

A pain-relieving pharmaceutically active substance is e.g. a non-steroidal anti-inflammatory drug (“NSAID”), or a local anesthetic, or a warming agent, e.g. capsaicin or vanillyl butyl ether—and preferably a NSAID.

NSAIDs are e.g. diclofenac, indomethacin, ibuprofen, piroxicam, acetylsalicylic acid (Aspirin®), naproxen, methyl salicylate, ketoprofen, tolfenamic acid, phenylbutazone, piroxicam, nimesulide, lumiracoxib, and pharmaceutically acceptable salts of any of said compounds. Preferred as NSAID is diclofenac or a pharmaceutically acceptable salt thereof. Especially preferred are diclofenac free acid, diclofenac sodium, diclofenac potassium, diclofenac diethylammonium and diclofenac epolamine, more especially diclofenac sodium and diclofenac diethylammonium, and in particular diclofenac sodium.

Local anesthetics are e.g. lidocaine, benzocaine, dibucaine or procaine.

Also combinations, e.g. of a NSAID and a warming agent, or of a NSAID and a local anesthetic, come into consideration as pain-relieving active substances.

The compositions of the invention may optionally include further excipients known in the art, for example fragrances/perfumes, antioxidants, e.g. butylhydroxytoluene, antimicrobial preservatives, e.g. benzyl alcohol, benzalkonium chloride or parabens, coloring agents or pH regulator components (sometimes required to optimize a drug's permeation).

The compositions of the invention typically form a discreet flexible transparent film. If desired, they can also be modified to form an opaque white or colored film by e.g. adding corresponding coloring agents.

In a special embodiment of the invention, the compositions for percutaneous administration of the invention, as defined hereinbefore or hereinafter, in addition comprise of from 0.5 up to 8% (w/w)—preferably of from 1 up to 6% (w/w), in particular of from 1.5 up to 4% (w/w)—of a compound selected from the group consisting of oleic alcohol, oleic acid, linoleic alcohol, linoleic acid, myristyl alcohol, dimethyl sulofoxide, thymol, menthol, preferably levomenthol, and any mixtures thereof. Especially preferred is a mixture of levomenthol and oleyl alcohol.

In another embodiment of the invention, the compositions for percutaneous administration of the invention, as defined hereinbefore or hereinafter, are devoid of ethyl acetate.

In a further embodiment of the invention, the compositions for percutaneous administration of the invention, as defined hereinbefore or hereinafter, are devoid of esters of C1-C10 alcohols with C1-C10 carboxylic acids.

In a still further embodiment of the invention, the compositions for percutaneous administration of the invention, as defined hereinbefore or hereinafter, are devoid of both ethyl acetate and salicylic acid.

In another embodiment of the invention, the compositions for percutaneous administration of the invention, as defined hereinbefore or hereinafter, are devoid of both esters of C1-C10 alcohols with C1-C10 carboxylic acids and salicylic acid.

In another embodiment of the invention, the compositions for percutaneous administration of the invention, as defined hereinbefore or hereinafter, are devoid of all of ethyl acetate, salicylic acid and cellulose nitrate.

In another embodiment of the invention, the compositions for percutaneous administration of the invention, as defined hereinbefore or hereinafter, are devoid of a keratolytically active agent, in particular devoid of a keratolytically active agent being defined as an agent which is suitable to effect the dissolution and detachment of korneocytes from the stratum corneum, and which keratolytically active agent is selected, for example, from the group consisting of retinoid receptor agonists, such as adapalene and retinoids, particularly tretinoin, isotretinoin, motretinide, tazarotene and/or retinol; urea; organic acids, particularly hydroxy carboxylic acids, especially glycolic acid, acetic acid, lactic acid and/or salicylic acid; and mixtures thereof.

Preferred embodiments of the invention are characterized by replacing in all definitions and embodiments of the invention hereinbefore and hereinafter (including the claims) “[composition . . . ] comprising” with ““[composition . . . ] consisting essentially of”.

Even more preferred embodiments of the invention are characterized by replacing in all definitions and embodiments of the invention hereinbefore and hereinafter (including the claims) “[composition . . . ] comprising” with ““[composition . . . ] consisting of”.

The compositions for percutaneous administration of the invention can be manufactured in a manner known per se, for example by conventional mixing and homogenization methods, and as described in the Examples section below.

The compositions for percutaneous administration of the invention show inter alia excellent long term efficacy, mechanical robustness and waterproofness as well as a high skin permeation of the drug, as far as desired, over a long period of time. Said beneficial properties can be demonstrated e.g. by the following tests:

(1) The mechanical properties of the films are tested by, in particular, measuring the tensile strength, the Young's modulus and the elongation of the film. Moreover, the films are tested e.g. in a shear test, a stress relaxation or an elastic deformation test.
(2) Film robustness is determined e.g. by oscillating a piece of gauze over glass slides on which 100 mg of a test composition have been evenly spread and allowed to dry at 50° C. for 10 min.
(3) Specific properties related to the application of the compositions of the invention that are tested are their spreadability, their resistance to water and their skin adhesion.
(4) Waterproofness is determined e.g. by evenly spreading a test composition on glass slides, allowing to dry it and weighing the glass slide with the dried film. The glass slides are immersed in a beaker of deionized water at 20° C. for 20 min. Then they are removed, dried in an oven at 50° C. and weighed again. Waterproofness is calculated from the weights of the glass slides before and after water treatment.
(5) In vitro skin retention of drug component: The skin levels of the drug are determined after application of the test composition on the skin surface after 24 h and within the epidermis after 24 h. In vitro diffusion cells using excised human epidermis are used. The test composition is applied to epidermal membrane and the amount of drug penetrating subsequently measured (HPLC and UV detection).

Another embodiment of the invention concerns the compositions for percutaneous administration of the invention, comprising at least one pain-relieving pharmaceutically active substance—preferably a non-steroidal anti-inflammatory drug, in particular diclofenac—for use in the treatment of pain.

Still another embodiment of the invention concerns the compositions for percutaneous administration of the invention comprising at least one pain-relieving pharmaceutically active substance—preferably a non-steroidal anti-inflammatory drug, in particular diclofenac—for use in the treatment of pain in limbs or joints (articulations) as well as back pain of a patient via forming a durable film at the site of pain thus enabling said pain-relieving pharmaceutically active substance—preferably a non-steroidal anti-inflammatory drug, in particular diclofenac—to penetrate through the skin over a long period of time. A “long period of time” typically means of from 8 hours up to 7 days, preferably of from 8 hours up to 4 days and in particular of from 8 hours up to 48 hours.

Especially, said compositions for percutaneous administration comprising at least one NSAID are suitable to treat ankle pain—e.g. ankle sprain—, wrist pain, knee pain—e.g. knee sprain or knee osteoarthritis—, elbow pain—e.g. tennis elbow or elbow osteoarthritis—, shoulder pain, finger pain—e.g. finger sprain or finger osteoarthritis such as thumb osteoarthritis, or back pain.

Another embodiment of the invention concerns the use of (a) ethyl acrylate/methyl methacrylate copolymer, (b) ethanol, isopropanol or a mixture thereof, (c) water, and (d) at least one non-steroidal anti-inflammatory drug—in particular diclofenac—for the manufacture of a composition for use in the treatment of pain.

The beneficial properties of said compositions for percutaneous administration comprising at least one NSAID are demonstrated e.g. by the following tests:

(1) in vitro cumulative permeation on human skin at 24 h (measured in μg diclofenac/cm² [area of composition applied]): The compositions of Examples 1-11 show a high in vitro cumulative permeation on human skin at 24 h.
(2) Pain relief: The compositions of Examples 1-11 show high pain relief when applied to patients suffering from ankle sprain or back pain, respectively.

The following examples are intended to illustrate the invention.

Example 1: Transparent, homogeneous gel comprising 40% (w/w) of Diclofenac sodium
Example 2: Transparent, homogeneous gel comprising 4.0% (w/w) of Diclofenac sodium
Example 3: Transparent, homogeneous gel comprising 4.0% (w/w) of Diclofenac sodium

| | Example 1 | Example 2 | Example 3 |
|---|---|---|---|
| | %(w/w) | %(w/w) | %(w/w) |
| Diclofenac sodium | 4.0 | 4.0 | 4.0 |
| Diclofenac | — | — | — |
| diethylammonium | | | |
| Eudragit ® NE40D | 30.0, | 30.0, | 30.0, |
| consisting of: | consisting of: | consisting of: | consisting of: |
| EA/MMA copolymer | 12.0 | 12.0 | 12.0 |
| water | 18.0 | 18.0 | 18.0 |
| Isopropanol | 66.0 | 65.5 | — |
| Ethanol, absolute | | | 66.0 |
| Levomenthol | — | 0.5 | — |
| Oleyl alcohol | — | — | — |
| Water, purified | — | — | — |
| Total | 100.0 | 100.0 | 100.0 |

(EA/MMA copolymer = ethyl acrylate/methyl methacrylate copolymer)

Example 4: Slightly opalescent gel comprising 4.6% (w/w) of Diclofenac sodium
Example 5: Transparent, homogeneous gel comprising 4.0% (w/w) of Diclofenac sodium
Example 6: Clear gel comprising 4.0% (w/w) of Diclofenac sodium

| | Example 4 | Example 5 | Example 6 |
|---|---|---|---|
| | %(w/w) | %(w/w) | %(w/w) |
| Diclofenac sodium | 4.6 | 4.0 | 4.0 |
| Diclofenac | — | — | — |
| diethylammonium | | | |
| Eudragit ® NE40D | 17.2, | 30.0, | 40.0, |
| consisting of: | consisting of: | consisting of: | consisting of: |
| EA/MMA copolymer | 6.9 | 12.0 | 16.0 |
| water | 10.3 | 18.0 | 24.0 |
| Isopropanol | 68.6 | 33.0 | 56.0 |
| Ethanol, absolute | — | 33.0 | — |
| Levomenthol | — | — | — |
| Oleyl alcohol | — | — | — |
| Water, purified | 9.6 | — | — |
| Total | 100.0 | 100.0 | 100.0 |

Example 7: Clear gel comprising 5.1% (w/w) of Dicofenac diethylammonium
Example 8: Gel comprising 4.0% (w/w) of Diclofenac sodium
Example 9: Viscous gel comprising 4.0% (w/w) of Diclofenac sodium

| | Example 7 | Example 8 | Example 9 |
|---|---|---|---|
| | %(w/w) | %(w/w) | %(w/w) |
| Diclofenac sodium | — | 4.0 | 4.0 |
| Diclofenac | 5.1 | — | — |
| diethylammonium | | | |
| Eudragit ® NE40D, | 27.5, | 40.0, | 50.0, |
| consisting of: | consisting of: | consisting of: | consisting of: |
| EA/MMA copolymer | 11.0 | 16.0 | 20.0 |
| water | 16.5 | 24.0 | 30.0 |
| Isopropanol | 67.4 | 53.5 | 43.5 |
| Levomenthol | — | 0.5 | 0.5 |
| Oleyl alcohol | — | 2.0 | 2.0 |
| Water, purified | — | — | — |
| Total | 100.0 | 100.0 | 100.0 |

Example 10: Viscous liquid comprising 4.6% (w/w) of Diclofenac sodium
Example 11: Viscous liquid comprising 4.6% (w/w) of Diclofenac sodium

| | Example 10 | Example 11 |
|---|---|---|
| | %(w/w) | %(w/w) |
| Diclofenac sodium | 4.6 | 4.6 |
| Diclofenac | — | — |
| diethylammonium | | |
| Eudragit ® NE40D, | 10.0, | 10.0, |
| consisting of: | consisting of: | consisting of: |
| EA/MMA copolymer | 4.0 | 4.0 |
| water | 6.0 | 6.0 |
| Isopropanol | 75.4 | 85.4 |
| Levomenthol | — | — |
| Oleyl alcohol | — | — |
| Water, purified | 10.0 | — |
| Total | 100.0 | 100.0 |

The formulations of the Examples 1-11 are manufactured as follows: Dissolve diclofenac salt, levomenthol and oleyl alcohol in a mix of Eudragit NE40D and additional water (if present). Then add isopropanol and/or ethanol under strong mixing (with gel formation, except Examples 10-11).

1. A composition for percutaneous administration of a physiologically active agent comprising
(a) ethyl acrylate/methyl methacrylate (EA/MMA) copolymers,
(b) ethanol, isopropanol or a mixture thereof,
(c) water, and
(d) at least one physiologically active agent.

2. A composition according to claim 1, wherein (b) is isopropanol.

3. A composition according to claim 1, wherein the at least one physiologically active agent (d) is selected from the group consisting of pain-relieving pharmaceutically active substances, antihistamines, antifungals and nicotine.

4. A composition according to claim 3, wherein the at least one physiologically active agent (d) is diclofenac or a pharmaceutically acceptable salt thereof.

5. A composition according to claim 1, which contains comprises EA/MMA copolymers (a)—calculated in pure form—in an amount of from 2% up to 30% (w/w).

6. A composition according to claim 1, which comprises component (b) in an amount of from 35% up to 90% (w/w).

7. A composition according to claim 1, which comprises water (c) in an amount of from 5% up to 40% (w/w).

8. A composition according to claim 1, which comprises EA/MMA copolymers (a)—calculated in pure form—and component (b) in a weight ratio of from 1:1 up to 1:45.

9. A composition according to claim 1, which in addition comprises from 0.5 to 8% (w/w) of a compound or compounds selected from the group consisting of oleic alcohol, oleic acid, linoleic alcohol, linoleic acid, myristyl alcohol, dimethyl sulofoxide, thymol, menthol, and any mixtures thereof.

10. A composition according to claim 3, comprising at least one pain-relieving pharmaceutically active substance.

11. A composition according to claim 3, comprising at least one pain-relieving pharmaceutically active substance.

12. A method for treating pain in a subject in need thereof comprising percutaneous administration of a composition comprising:
(a) ethyl acrylate/methyl methacrylate copolymer,
(b) ethanol, isopropanol or a mixture thereof,
(c) water, and
(d) at least one non-steroidal anti-inflammatory drug.

13. A composition according to claim 5, which comprises component (b) in an amount of from 35% up to 90% (w/w).

14. A composition according to claim 1, which in addition comprises from 0.5 to 8% (w/w) of a mixture of levomenthol and oleyl alcohol.

15. A composition according to claim 2, comprising component (b) in an amount of from 35% up to 90% (w/w), and additionally comprising from 0.5 to 8% (w/w) of a mixture of levomenthol and oleyl alcohol.

16. A composition according to claim 13, which in addition comprises from 0.5 to 8% (w/w) of a mixture of levomenthol and oleyl alcohol.

17. A composition according to claim 15, comprising as component (d), diclofenac or a pharmaceutically acceptable salt thereof.
